(19) United States
(12) Patent Application Publication
(10) Pub. No.: US 2023/0082418 A1
(43) Pub. Date: Mar. 16, 2023

(54) PROTECTIVE GARMENT

(71) Applicant: HANES INNERWEAR AUSTRALIA PTY LTD, Kew (AU)

(72) Inventor: Heidi MELLOS, Kew (AU)

(21) Appl. No.: 17/802,236

(22) PCT Filed: Feb. 26, 2021
(86) PCT No.: PCT/AU2021/050164

§ 371 Date: Aug. 25, 2022

(30) Foreign Application Priority Data
Feb. 26, 2020 (AU) 2020900547

(51) Int. Cl. A41D 31/12; A41D 13/00; A61F 13/15; A41B 9/12

(52) U.S. Cl. CPC A41D 31/12; A41D 13/0015; A61F 13/15268; A41B 9/12; A61F 2013/15121; A61F 2013/15195; A61F 2013/15276; A61F 2013/15284; D06M 2101/06

(57) ABSTRACT

A protective garment, gussets for protective garments, and individual gusset layers, being a moisture wicking layer, a core moisture absorbing layer and a moisture impermeable layer, and combinations thereof. The moisture wicking layer including a double knit fabric wherein a technical face of the fabric forms an eyelet mesh utilising both hydrophilic and hydrophobic yarns and a technical back of the fabric forms an interlock utilising only hydrophilic yarns. The moisture absorbing layer including a sinker terry fabric knit comprising sinker loops with plush loops made form synthetic fibres treated with a hydrolysing enzyme that splits the fibres into fibrils. The moisture impermeable layer including a base fabric knit laminated with a polyurethane sheet.

### FIELD OF THE INVENTION

The present invention relates to a protective garment, in particular an undergarment or a garment including a gusset, such as a bodysuit or swimsuit, a reusable gusset that can be incorporated into a garment, and separate gusset layers, and associated methods of construction.

### BACKGROUND OF THE INVENTION

Sanitary products are used to absorb bodily discharge, including menstrual flow and incontinence leaks. Many of these products are disposable, for example sanitary pads, tampons and incontinence pads. Many products that are used to absorb large flows are bulky and uncomfortable for the wearer.

Some “period underwear” for everyday use has been released that is comfortable, but is designed to act as a back up garment in the case of leaks due to tampon, sanitary pad, or menstrual cup failures. Such failures often occur on heavy flow days or during changes in body position, such as whilst sleeping.

Disposable products are environmentally hazardous and there is desire to reduce waste products associated with these uses. Additionally, there is a greater risk of Toxic Shock Syndrome associated with products that are worn inside the vagina, such as tampons and menstrual cups. These products are required to be changed or emptied regularly to reduce the associated risks. However, external products, such as sanitary pads are associated with discomfort, a “plasticky feel”, a feeling of constant dampness, the need for regular changing and concerns around odour management.

The present invention seeks to provide a protective garment, or gusset that can be incorporated into a garment, or gusset layers, which are reusable and offer an alternative to existing products.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

According to a first aspect, the present invention provides a moisture wicking layer for incorporation into a gusset for a protective garment, the gusset layer being a double knit fabric constructed from yarn, wherein a first portion of the yarns are hydrophilic and a second portion of the yarns are hydrophobic, whereby a technical face of the fabric forms an eyelet mesh utilising both hydrophilic and hydrophobic yarns and a technical back of the fabric forms an interlock utilising only hydrophilic yarns, and whereby the hydrophobic yarns only outwardly present to the technical face of the fabric, and the technical face forms eyelet openings formed by a tuck stitch being held in a repeated pattern to expose the underlying hydrophilic yarns of the technical back to wick moisture away from the technical face.

The moisture wicking layer preferably comprises absorbent cotton yarn.

Approximately 25% of the cotton yarn is preferably chemically treated to make it hydrophobic. The eyelet mesh may be made with 50% hydrophilic and 50% hydrophobic yarns. The interlock is preferably made with 100% hydrophilic yarn.

The technical face may be a 2 course repeat of 1 course hydrophilic yarn×1 course hydrophobic yarn.

According to a second aspect, there is provided a gusset for a protective garment, that includes a moisture wicking layer according to the first aspect of the invention together with a moisture absorbing layer and a moisture impermeable layer.

According to a third aspect, there is provided a protective garment with a gusset according to the second aspect of the invention.

According to a fourth aspect, the present invention provides a moisture absorbing layer for incorporation into a gusset for a protective garment, the gusset layer having a sinker terry fabric knit comprising sinker loops with plush loops protruding from the fabric knit on at least one side, wherein the plush loops are made from synthetic fibres treated with a hydrolysing enzyme that splits the fibres into fibrils.

The sinker terry fabric knit is constructed using a sinker terry knitting machine as opposed to a single construction knitting machine where loop formation is created by miss stitch.

The moisture absorbing layer may have plush loops protruding from both sides. The moisture absorbing layer may have plush loops protruding from only one side. When the plush loops protrude from only one side, the other side may be laminated with a moisture impermeable sheet, for example a polyurethane sheet.

The sinker loops may be made from polyester yarn. The plush loops may be made with a polyester/nylon yarn, which may be 80% polyester and 20% nylon. The height of the plush loops may be in the range of 1.5 mm to 3.5 mm, more preferably approximately 2.5 mm.

The hydrolysing enzyme may be sodium hydroxide. The splitting may result in the fibres being split into 4-10 times as many fibrils.

The sinker terry fabric knit is preferably 100% hydrophilic.

According to a fifth aspect, there is provided a gusset for a protective garment, that includes at least one moisture absorbing layer in accordance with the fourth aspect of the invention together with a moisture wicking layer and a moisture impermeable layer. The moisture wicking layer may be according to the first aspect of the invention.

There may be provided a gusset that includes more than one moisture absorbing layer according to the fourth aspect.

According to a sixth aspect, there is provided a protective garment with a gusset according to the fifth aspect of the invention.

According to a seventh aspect, the present invention provides a moisture impermeable layer for incorporation into a gusset for a protective garment, the gusset layer including a base fabric knit laminated with a polyurethane sheet.

The base fabric knit may be a polyester jersey.

According to an eighth aspect, there is provided a gusset for a protective garment, that includes a moisture impermeable layer according to the seventh aspect of the invention together with a moisture wicking layer and a moisture absorbing layer.

The moisture impermeable layer may extend further rearwardly than the moisture wicking layer or the moisture absorbing layer.

According to a ninth aspect, there is provided a gusset for a protective garment, that includes a moisture wicking layer according to the first aspect and a moisture impermeable layer according to the seventh aspect, together with a moisture absorbent layer.

According to a tenth aspect, there is provided a gusset for a protective garment, that includes a moisture absorbing layer according to the fourth aspect and a moisture impermeable layer according to the seventh aspect, together with a moisture wicking layer. The moisture wicking layer may be according to the first aspect.

According to an eleventh aspect, there is provided a garment having a gusset according to the eighth aspect.

According to a twelfth aspect, there is provided a garment having a gusset according to the ninth aspect.

According to a thirteenth aspect, there is provided a garment having a gusset according to the tenth aspect.

According to a fourteenth aspect, there is provided a garment having a body fabric that is a cotton/elastane jersey knit which has an odour absorption property inherent in filament of the elastane yarn. The jersey knit preferably includes 7 or more % of elastane. The garment may incorporate a gusset according to any of the above-mentioned aspects.

According to a fifteenth aspect, there is provided a garment having a body fabric that is a recycled polyester/elastane jersey knit which has an odour absorption property inherent in the filament of the elastane yarn. The jersey knit preferably includes 23 or more % elastane. The garment may incorporate a gusset according to any one of the above-mentioned aspects.

A garment made in accordance with any of the above aspects, may include a waist band and/or leg bands. Either or both of these bands may be treated or coated with a durable water repellent (DWR) to counteract compression leakage. Additionally, the thread used for sewing the gusset to the body may be coated with DWR to safe guard against compression leakage at the seams.

### BRIEF DESCRIPTION OF THE DRAWINGS

Further aspects of the present invention and further embodiments of the aspects described in the preceding paragraphs will become apparent from the following description, given by way of example and with reference to the accompanying drawings, in which:

FIG. 1 is a representative view of a protective garment according to an embodiment on a wearer;

FIG. 2a is a front view of an undergarment according to an embodiment;

FIG. 2b is a rear view of the undergarment of FIG. 2a;

FIG. 3a is a front view of an undergarment according to another embodiment;

FIG. 3b is a rear view of the undergarment of FIG. 3a;

FIG. 4a is a front view of an undergarment according to another embodiment;

FIG. 4b is a rear view of the undergarment of FIG. 4a;

FIG. 5a is a front view of an undergarment according to another embodiment;

FIG. 5b is a rear view of the undergarment of FIG. 5a;

FIG. 6 is a top view of a gusset according to an embodiment;

FIG. 7 is an exploded representative view of the layers of a gusset according to an embodiment for light/moderate flow;

FIG. 8 is an exploded representative view of the layers of a gusset according to an embodiment for heavy flow;

FIG. 9a is a representative drawing of a section of eyelet mesh knitting on the first face of a moisture wicking layer of the first aspect of the invention;

FIG. 9b is a representative drawing of a section of interlock knitting on the second face of a moisture wicking layer of the first aspect of the invention;

FIG. 9c is a representative drawing of a larger section of eyelet mesh knitting;

FIG. 9d is a representative drawing of a section of the eyelet mesh knitting on the first face combined with a section of the interlock knitting on the second face to form the wicking layer;

FIG. 10a is a representative drawing of a section of knitting of a moisture absorbing layer of the second aspect of the invention;

FIG. 10b is a representative drawing of a side view of a section of knitting of a moisture absorbing layer of the second aspect of the invention;

FIG. 11a and FIG. 11b are representative images of a yarn fibre before and after splitting;

FIG. 12a and FIG. 12b are microscopic images of yarn fibres before and after splitting;

FIG. 13a is a representative drawing of a section of knitting on the first face of a moisture impermeable layer of the third aspect of the invention;

FIG. 13b is a representative exploded view of the moisture impermeable layer of the third aspect of the invention;

FIG. 14a is a representative view of a section of knitting on the first face of the body fabric; and

FIG. 14 b is a representative view of a section of knitting on the second face of the body fabric.

### DETAILED DESCRIPTION OF THE EMBODIMENTS

The embodiments illustrated and described are in relation to period underwear and gussets for use in period underwear. However, it will be appreciated that the underlying inventive concepts of the gusset layers can be tailored for use in other protective garments, such as incontinence underwear, reusable toddler nappies and the like.

Whilst the underwear illustrated are briefs, the protective gussets may be incorporated into other garments that sit against the skin on the lower half of the body, for example swimwear or bodysuits.

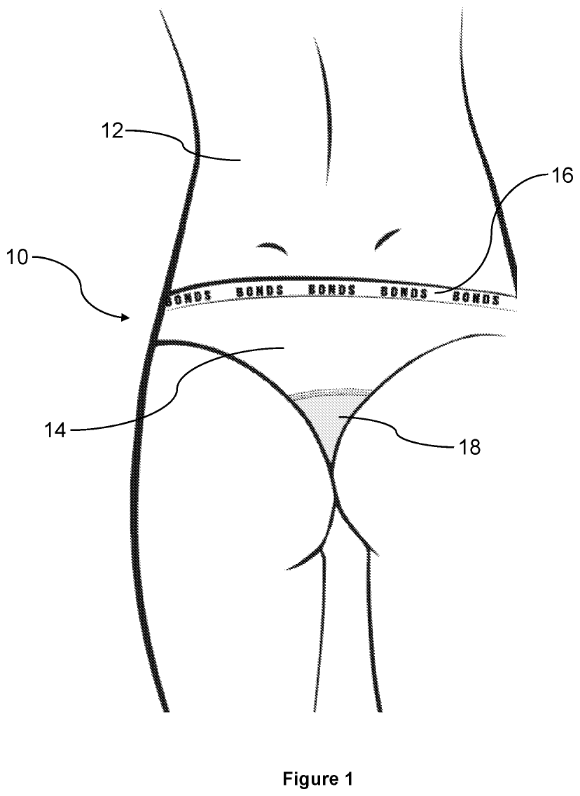

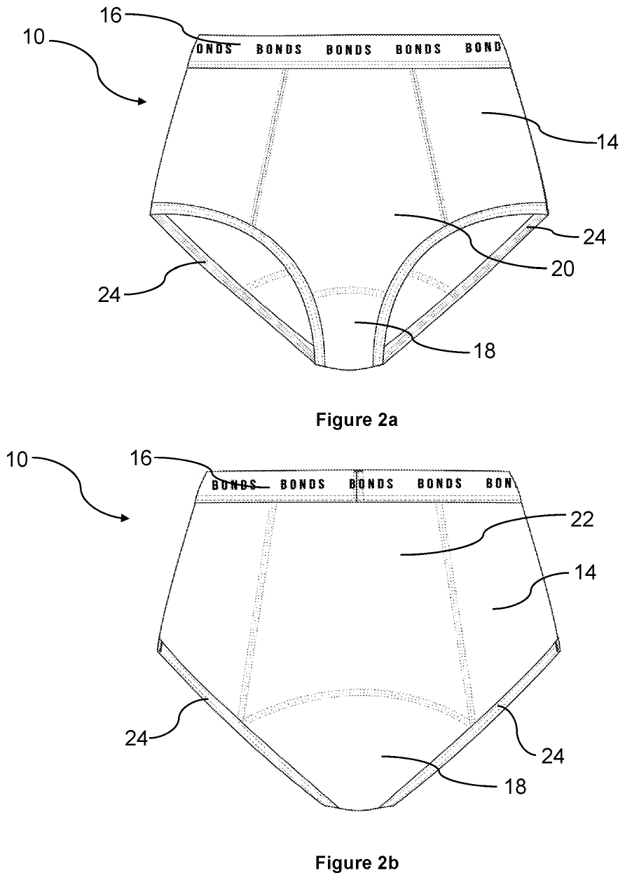

FIG. 1 illustrates a protective garment 10 on a wearer 12. The briefs 10 have a body portion 14 that sit around wearers hips below a waistband 16. Extending over at least the crotch region is a gusset 18. As shown in FIGS. 2a and 2b, the body portion 14 may include a front panel 20 and a rear panel 22. Extending around the leg openings are leg bands 24.

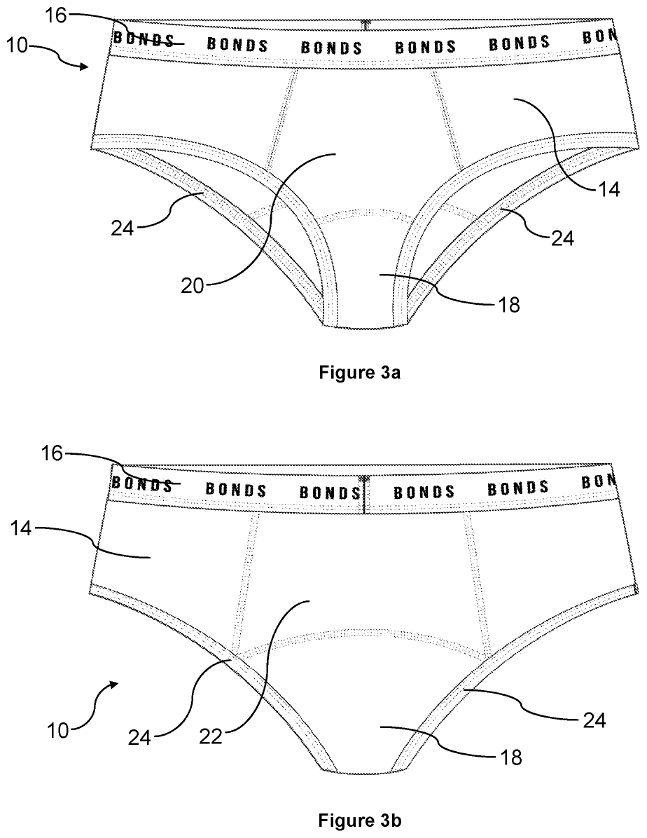

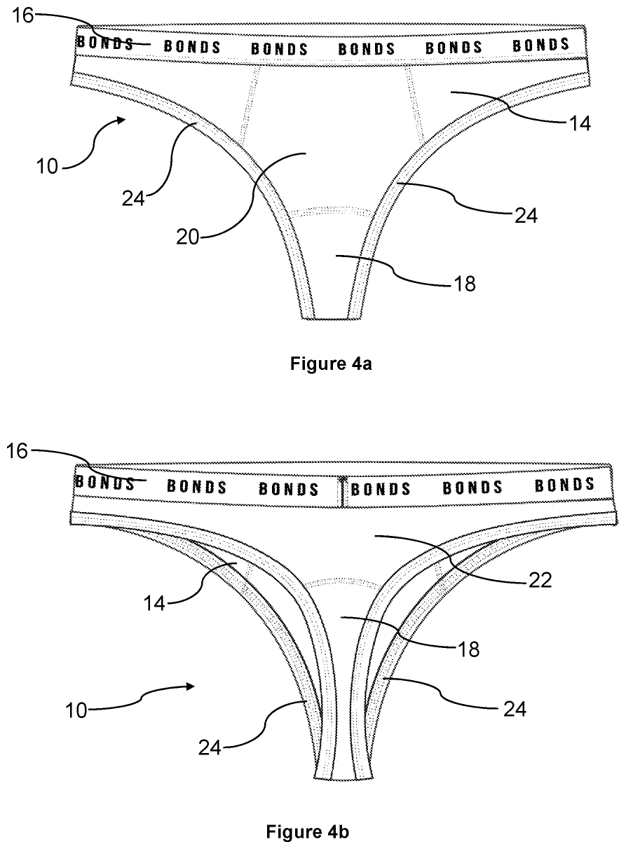

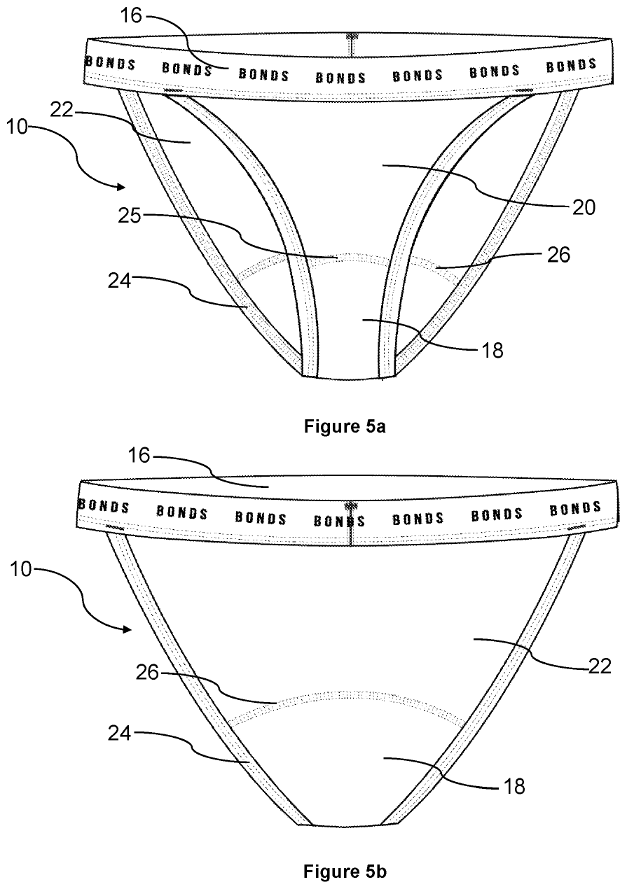

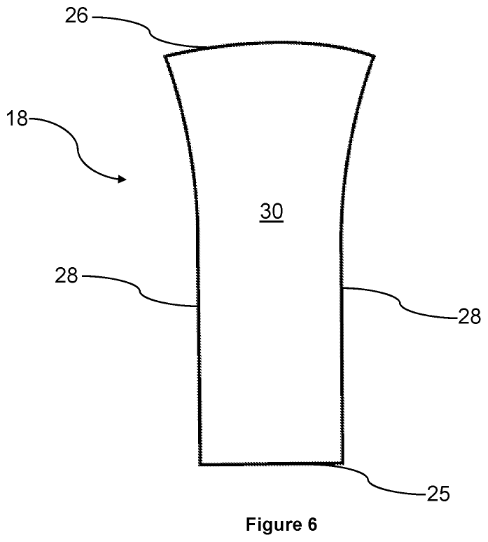

The shape of the briefs shown in FIGS. 2a and 2b are a Hi Top brief. FIGS. 3a and 3b illustrate a boyleg brief, FIGS. 4a and 4b illustrate a G-string brief and FIGS. 5a and 5b illustrate a tanga brief. However it will be appreciated that different shaped briefs can incorporate the inventive gusset. Like reference numerals have been used across the different embodiments illustrated. A similar or slightly altered shaped gusset 18 can be used across each of the different styles of briefs.

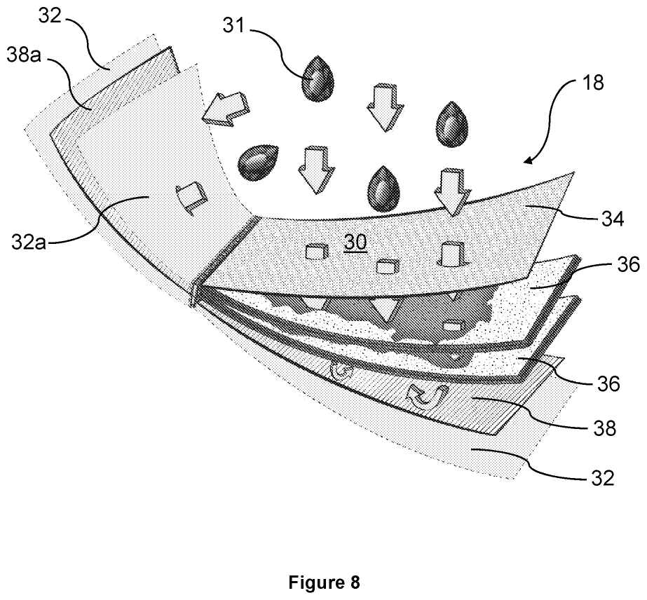

An example gusset shape is illustrated in FIG. 8. The gusset 18 includes a front edge 25, a rear edge 26 and two sides 28. The gusset 18 has a top surface 30 that sits against the wearer's skin in the crotch region. Turning back to FIG. 5a, it can be seen that the gusset 18 can be stitched to the body portion 14 across the front edge 25 and the rear edge 26. The two sides 28 of the gusset can also be stitched to the body portion along the edges of the leg openings before the leg bands 24 are affixed or the leg bands 24 can be used to affix the sides of the gusset to the body portion. The leg bands 24 and the thread used to stitch the gusset may be coated with a durable water repellent (DWR) to prevent wicking of blood to the edges and safe guard against compression leakage.

The gusset 18 is flared towards the rear edge 26 to minimise back bleeding over the edge. The gusset 18 may also be slightly flared towards the front edge 25 to minimise the potential for fluid to gush over the front.

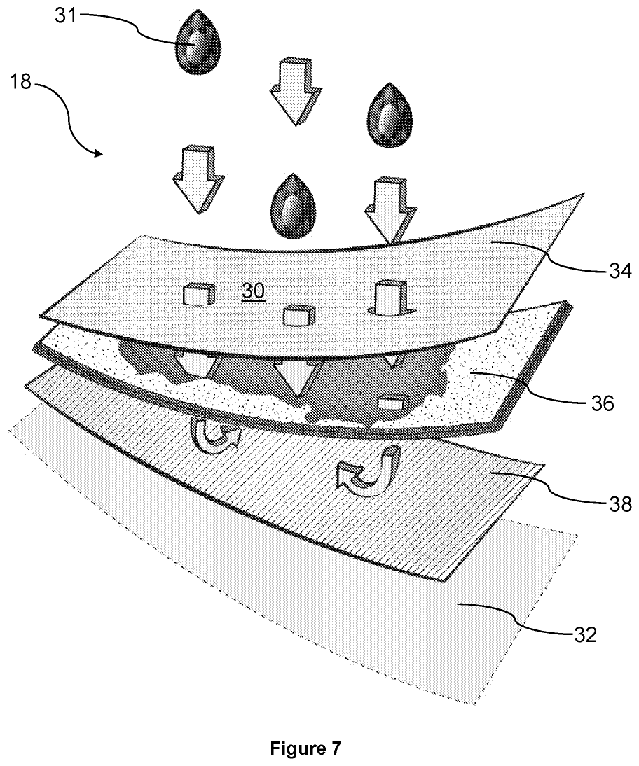

FIG. 7 illustrates a representative exploded view of a gusset 18 made for light or moderate flow of fluid 31, such as blood or urine. Light to moderate flow is specified in standards as being capable of holding 5-10 ml of fluid.

The gusset 18 is made from three layers, which are positioned inside of the body fabric layer 32 that is used to create the body portion 14 of the garment 10. In FIG. 7 the body fabric layer 32 has been illustrated as a rectangle for ease of understanding, but it will be appreciated that this layer extends outside of the broken line periphery to form the shape of the garment.

The top layer 34 is a moisture wicking layer that has a top surface 30 that sits against the wearer. The wicking layer 34 pulls fluid away from the user's skin to the core layer 36. The core layer is moisture absorbing and holds the fluid during use. The bottom layer 38 of the gusset is a moisture impermeable layer that ensures fluid does not escape onto the fabric layer 32.

FIG. 8 is similar to FIG. 7, such that it illustrates a representative exploded view of a gusset 18, however this version is made for a heavy or overnight flow. Heavy to overnight flow is specified in standards as being capable of holding 15-20 ml of fluid. For completeness, in embodiments not illustrated or described to be used for incontinence purposes, the holding volume required is 40 ml.

In this embodiment, the top moisture wicking layer 34 is the same as the previous embodiment. To increase absorbency, there is provided two core layers 36. To reduce the chance of back bleeding staining clothing, the bottom layer 38 of moisture impermeable material has a portion 38a that extends further rearwardly than the other gusset layers. An additional internal portion of body fabric layer 32a is provided over the portion 38a to provide a comfortable surface material against the wearer's skin. This body fabric layer 32a is illustrated as rectangular, but is more likely to extend all the way to the waistband 16 and form the rear panel 22, as shown in FIG. 2b. The body fabric layer has been provided in this area, rather than another section of the top wicking layer 34, so as not to encourage wicking towards this area. However, it will be appreciated that this rear panel 22 could be comprised of a wicking layer. The core layer 36 is limited to a defined crotch region, rather than extending further rearwardly in order to minimise discomfort to the wearer that comes with bulky products that extend too far rearwardly.

The details of the individual gusset layers, being a moisture wicking layer, a core moisture absorbing layer and a moisture impermeable layer, will now be described in relation to each of their own novel features. However, it will be appreciated that each of the individual gusset layers disclosed below may be exchanged with other types of moisture wicking layer, core moisture absorbing layer and moisture impermeable layer, to produce a novel gusset, and different combinations of individual layers are contemplated.

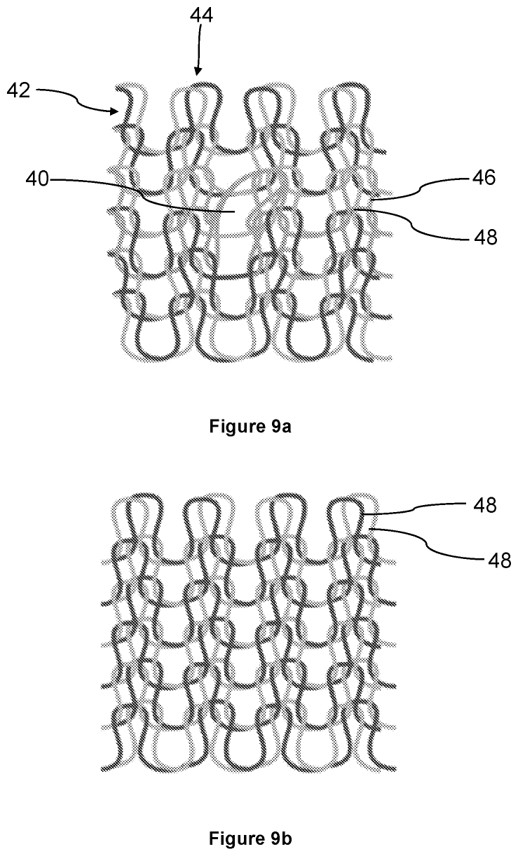

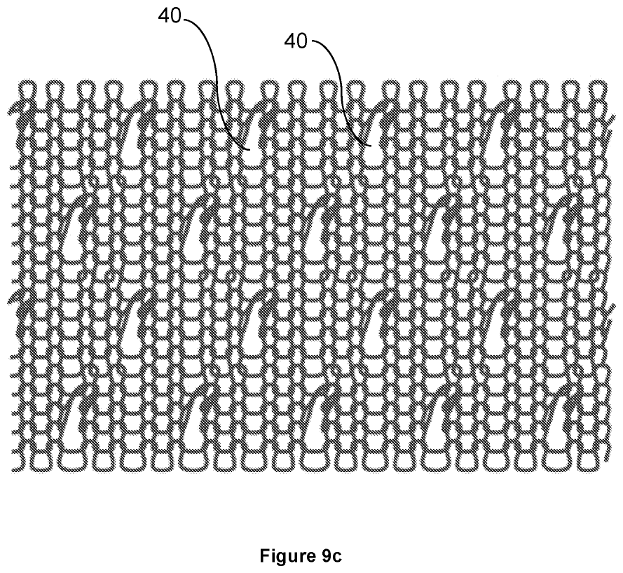

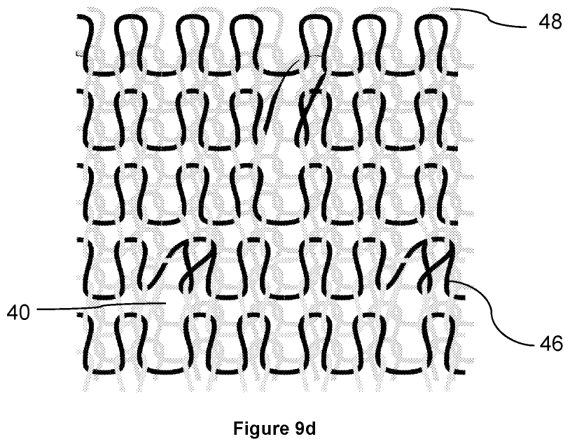

FIGS. 9a through 9d illustrate a representative knitting pattern for the top moisture wicking layer 34. This layer has moisture repellent cotton yarns blended with absorbent cotton yarns to create channels on the fabric surface that allows moisture to move away from the skin and keep the area cool and dry. The knitting is done using a combination of hydrophobic yarn and hydrophilic yarn. The yarn is a natural cotton, which is naturally hydrophilic and attracts moisture. A portion of the cotton yarns are chemically treated to make them hydrophobic. One preferred treatment is known as TransDry® Technology by CottonWorks™. The hydrophobic and hydrophilic yarns are double knit to create a different face on each side of the fabric.

The technical face that sits against the wearer's skin is an eyelet mesh where the yarns alternate per course equally between the hydrophobic and hydrophilic yarns. The technical back that sits against the core layer, is an interlock, which only uses hydrophilic yarn. In this construction, the eyelets on the technical face create openings by a tuck stitch being held in a repeated pattern to expose the underlying hydrophilic yarns of the technical back and encourage the moisture, in particular blood, to pass through the eyelet openings and wick through to the back. The combination of 50% of the yarns on the technical face being hydrophobic and the concentration of hydrophilic wicking yarns on the technical back, discourage moisture from returning to the technical face. The use of 50% of the yarns on the technical face being hydrophilic, rather than 100% hydrophobic, encourages increased wicking across a greater surface area.

FIGS. 9a and 9d illustrate the technical face of the moisture wicking layer, whereby the eyelets 40 are visible. The knitting terminology for a single transition of yarn across knitting needles is called a course 42, and runs horizontally across the fabric. A vertical line of stitches is called a wale 44, and when combined with a course creates a knit construction. The technical face is created as an eyelet jersey fabric. In eyelet jersey, a tuck stitch is held, in this example for 3 courses, to create an eyelet 40, being a mesh hole in the technical face of the fabric. A tuck stich is a commonly understood knitting technique to one skilled in the art, including when used on a double knit construction. The novelty of this construction is that it is only the hydrophobic yarn that is held. In this embodiment, it is the hydrophobic yarn 46 that is held every “even” course (2/4/6/8/10/12), and hydrophilic yarn 48 knitted every “odd” course (1/3/5/7/9/11) in a pattern repeat of 6 wales×12 courses.

In a double knit construction, two sets of needles (dial and cylinder) are “gaited”—spaced in relation to one another, which means needles can be used at the same time to create double knit fabrics. The technical back shown in FIG. 9b presents a standard interlock, a “gaited” construction of uniform stitches. All yarns 48 used on the technical back are hydrophilic. Thus, the fabric of the moisture wicking layer uses approximately 75% hydrophilic yarns and 25% hydrophobic yarns.

The unique way that the hydrophobic and hydrophilic yarns are knitted create channels for moisture to move away from the skin and directional one-way wicking is achieved. For the technical face sitting against the skin, 50% of the surface repels fluid such that it is guided to the other 50% of yarn, which draws it to the back face. As the back face is 100% hydrophilic yarn, the wicking is always away from the skin. This layer may also include an anti-stain repellent finish to increase the washability of the garments. By having this 50/50 split of hydrophobic/hydrophilic yarn against the wearer's skin, rather than 100% hydrophobic, the instance of moisture pooling on the surface is greatly reduced. By incorporating hydrophobic yarns, rather than being 100% hydrophilic, prevents the moisture from backflowing to the surface against the wearer's skin once it has wicked to the back face.

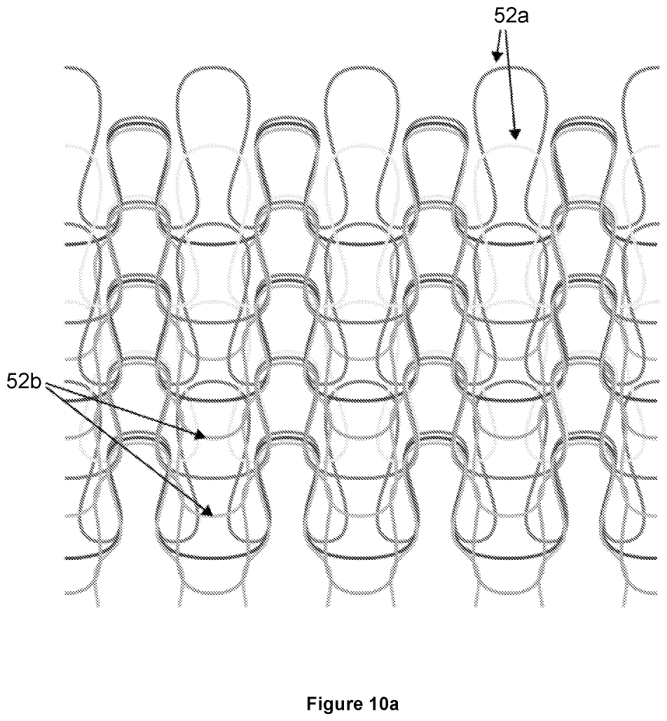

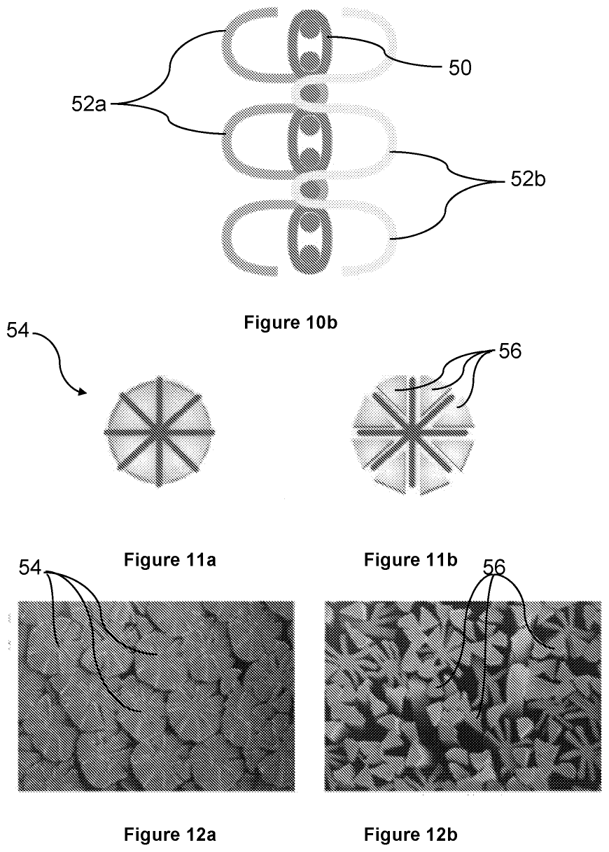

FIGS. 10a and 10b show representations of the construction of the core moisture absorbing layer. A large number of known period underwear utilise natural fibres such as wool or bamboo to construct an absorbent core, as these fibres are naturally hydrophilic and attract fluid. However, fluid moves into the fibres themselves whereby they swell to become very bulky, lumpy and heavy, which can be uncomfortable for the wearer. Synthetic fibres like polyester and nylon don't break the surface tension of fluid, so don't absorb as well as natural fibres, but they do allow a product to be made that is much thinner and less heavy. The present inventors have created a core layer out of synthetic fibres that provides similar absorbency to natural fibres, without some of the disadvantages of natural fibres.

In the core moisture absorbing layer, terry looped pile has been used, which acts like a sponge. The terry looped pile is created using a sinker terry knitting machine which is capable of varying the loop height and creating a high density pile. A fabric knit is created of sinker loops 50 with plush loops 52 protruding from at least one side. A person skilled in the art will appreciate how sinker terry loop pile is created as opposed to single construction knitting where loops are formed by miss stitches.

FIGS. 10a and 10b illustrate a double face looped pile. Front loops 52a protrude from one face and back loops 52b protrude from the other face. The double face looped pile may be used on light to medium flow undergarments, where an absorbency capacity equates to 4 tampons. Light flow garments could use a single face looped pile to obtain the required absorbency and make the gusset thinner, where an absorbency capacity equates to 3 tampons. Additionally, two double face looped pile layers could be used for overnight flow. An alternative for high flow would be one double face layer and one single face layer.

In one embodiment, the yarn used for the sinker loops 50 is 100% polyester, whilst the yarn used for the plush loops is 80% polyester and 20% nylon. The plush loop height may be in the range of 1.5 mm to 3.5 mm, but more preferably around 2.5 mm.

To make the synthetic fibres of the plush loops more absorbent they are treated with a hydrolysing enzyme that splits the fibres into fibrils. Sodium hydroxide is used before dying the yarn to split the polyester and nylon fibres by at least partially dissolving and removing the nylon. In one example, the yarn is split from 90D/36F (36 fibres) into approx. 90D/300F (300 fibres or fibrils). The wording fibrils is being used to describe the individual threads into which a fibre can be split. Example images of fibre splitting are shown in FIGS. 11a, 11b, 12a and 12b. FIG. 11a shows a single fibre 54 and FIG. 11b shows fibre 54 after splitting into eight fibrils 56. FIG. 12a shows a plurality of fibres 54 making up a yarn, with FIG. 12b showing the yarn after splitting the fibres 54 into fibrils 56. The fibres 54 may remain intact in the centre, with just the edges splitting, as shown in FIG. 12b. This may occur when the nylon does not fully dissolve.

In one example embodiment, a single face terry looped pile was made using a 30 inch diameter, 16 gauge, sinker terry knitting machine manufactured by Tien Yang Knitting Machinery Co. and a combination of polyester and nylon blended yarns. The single face terry looped pile was subjected to a micro-fibre splitting treatment with an alkali solution of 12 grams sodium hydroxide per litre at 98 degrees Celsius for 45 minutes. The treatment at least partially dissolved and removed the nylon core of the blended yarn used in the knitting of the single face terry looped pile resulting in the formation of a plurality of fibrils 56. Next, the single face terry looped pile was dyed in a circular dying machine with a solution of: a disperse dye, Dianix Blue ACE manufactured by Dystar, at 0.003% on weight of fabric (o.w.f.), 0.5 grams per litre of a levelling agent, LM-0850 manufactured by Nicca Chemical Co., 0.25 grams per litre of acetic acid, and hydrophilic agent ASR, manufactured by Rudolf, at 2.5% o.w.f. The dyeing process was completed at 130° C. for 30 minutes.

In another example embodiment, a double face terry looped pile was made using a 30 inch diameter, 16 gauge, terry knitting machine manufactured by Tien Yang Knitting Machinery Co. and a combination of polyester and nylon blended yarns. The double face terry looped pile was subjected to a micro-fibre splitting treatment with an alkali solution of 12 grams sodium hydroxide per litre at 98 degrees Celsius for 45 minutes. The treatment at least partially dissolved and removed the nylon core of the blended yarn used in the knitting of the double face terry looped pile resulting in the formation of a plurality of fibrils 56. Next, the double face terry looped pile was dyed in a circular dying machine with a solution of: a disperse dye, Dianix Blue ACE manufactured by Dystar, at 0.003% o.w.f., 0.5 grams per litre of a levelling agent, LM-0850 manufactured by Nicca Chemical Co., 0.25 grams per litre of acetic acid, and hydrophilic agent ASR, manufactured by Rudolf, at 2.5% o.w.f. The dyeing process was completed at 130° C. for 30 minutes. The core layer 36 is relatively thin, flexible and can absorb five times its own weight. Absorption testing was carried out on a double face looped pile core layer and findings included a mean liquid absorbency time of 17.21 seconds and a mean porcine blood absorptive capacity of 29 ml. Also, the core layer was found to be quick drying, such that the garments will be user friendly in terms of washing and drying for reuse.

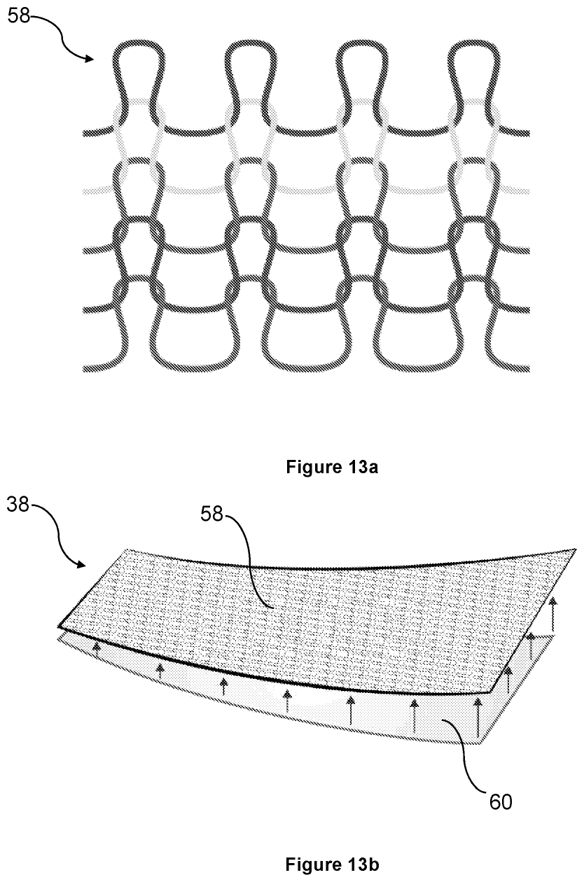

The moisture impermeable layer 38 is shown in FIGS. 13a and 13b. A base knit 58 is a jersey construction, as shown in FIG. 13a, using a polyester yarn. Polyester yarn is somewhat hydrophobic due to grease, however the grease is removed during finishing of the fabric so that the base knit becomes hydrophilic to allow lamination with a polyurethane (PU) sheet 60. The lamination techniques used are common to those skilled in the art and are not explained in detail here.

The PU sheet 60 is stretchable and has high breathability. The benefit of laminating the PU sheet to a base knit is that it makes the layer less “plasticky” in feel, movement and noise. It also creates a strong base to make the PU sheet more durable allowing for repeated washing minimising degradation or ripping of the PU sheet.

Hydrostatic pressure testing was performed on sample moisture impermeable layers to determine the resistance to fluid penetration. The samples were subjected to a mean kPa of 250 and no droplets passed through the samples, with the sample bursting when the pressure exceeded 250 KPa. The moisture impermeable layer 38 therefore successfully prevents leakage of fluid to the body fabric 32.

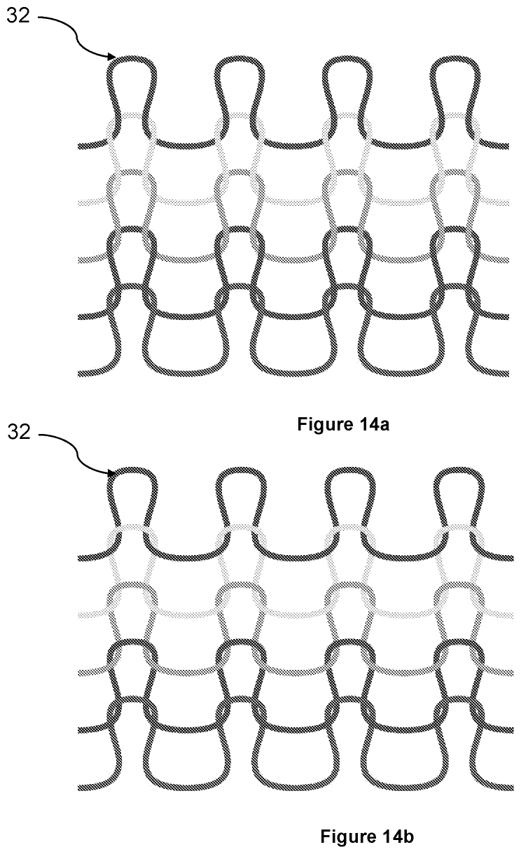

The knitting pattern of the body fabric 32 is illustrated in FIGS. 14a and 14b. The technical face of standard jersey knit has a “flat” surface, whereas, the technical back is denoted by a “textured/wavy” appearance. In one example, a combination of cotton/elastane is used, with 93% cotton yarn and 7% spandex yarn to provide a cotton-rich fabric with 4-way stretch, providing freedom to move and elevated comfort. Odour absorption is an inherent part of the elastane yarn used, such yarn may be Roica™ CF by Asahi Kasei. The ionic effect at the filament surface makes it permanent and eliminates odour by absorbing their main sources i.e. ammonia, acetic acid, isovaleric acid or products of their bacterial decomposition. This allows the fabric to withstand wear and repeated washing without reducing the anti-odour effects. Alternatively, a combination of recycled polester/elastan jersey knit is used, which has an odour absorption property inherent in filament of the elastane yarn. The jersey knot preferably includes 23 or more % of elastane. A stain release finish is applied to either jersey knit in the case of non-black colours to ensure any blood migration that occurs during wear is easily washed out without staining. A suitable stain release formula may be: HPC (20 g/l), CDO (2 g/l), GSI (20 g/l) & PURE (20 g/l).

The present invention relates to individual novel gusset layers that can be used in different combinations to create gussets for use in protective garments. The combined benefits of the gusset layers results in a gusset that is relatively thinner, has high wicking and absorbency, superior leak resistance and a comfortable natural fibre surface that sits against the wearer's skin.

It will be understood that the invention disclosed and defined in this specification extends to all alternative combinations of two or more of the individual features mentioned or evident from the text or drawings. All of these different combinations constitute various alternative aspects of the invention.

1. A moisture wicking layer for incorporation into a gusset for a protective garment, the gusset layer being a double knit fabric constructed from yarn, wherein a first portion of the yarns are hydrophilic and a second portion of the yarns are hydrophobic, whereby a technical face of the fabric forms an eyelet mesh utilising both hydrophilic and hydrophobic yarns and a technical back of the fabric forms an interlock utilising only hydrophilic yarns, and whereby the hydrophobic yarns only outwardly present to the technical face of the fabric, and the technical face forms eyelet openings formed by a tuck stitch being held in a repeated pattern to expose the underlying hydrophilic yarns of the technical back to wick moisture away from the technical face.

2. A moisture wicking layer according to claim 1 comprising absorbent cotton yarn.

3. A moisture wicking layer according to claim 2, wherein approximately 25% of the cotton yarn is chemically treated to make it hydrophobic.

4. A moisture wicking layer according to claim 1, wherein the technical face is a 2 course repeat of 1 course hydrophilic yarn×1 course hydrophobic yarn.

5. A moisture wicking layer according to claim 1, wherein the tuck stitch being held is hydrophobic yarn.

6. A gusset for a protective garment, that includes a moisture wicking layer according to claim 1, together with a moisture absorbing layer and a moisture impermeable layer.

7. A protective garment with a gusset according to claim 6.

8. A moisture absorbing layer for incorporation into a gusset for a protective garment, the gusset layer having a sinker terry fabric knit comprising sinker loops with plush loops protruding from the fabric knit on at least one side, wherein the plush loops are made from synthetic fibres treated with a hydrolysing enzyme that splits the fibres into fibrils.

9. A moisture absorbing layer according to claim 8, wherein the plush loops protrude from one side or both sides.

10. A moisture absorbing layer according to claim 8, wherein, when the plush loops protrude from only one side, the other side may be laminated with a moisture impermeable sheet.

11. A gusset for a protective garment that includes at least one moisture absorbing layer in accordance with claim 8, together with a moisture wicking layer and a moisture impermeable layer.

12. A gusset for a protective garment according to claim 11, wherein the moisture wicking layer is according to claim 1.

13. A protective garment with a gusset according to claim 11.

14. A moisture impermeable layer for incorporation into a gusset for a protective garment, the gusset layer including a base fabric knit laminated with a polyurethane sheet.

15. A moisture impermeable layer according to claim 14, wherein the base fabric knit is a polyester jersey.

16. A gusset for a protective garment, that includes a moisture impermeable layer according to claim 14 together with a moisture wicking layer and a moisture absorbing layer.

17. A gusset according to claim 16, including a moisture wicking layer according to claim 1 and a moisture absorbent layer according to claim 8.

18. A gusset for a protective garment, that includes a moisture absorbing layer according to claim 8, and a moisture impermeable layer according to claim 14, together with a moisture wicking layer.

19. A gusset according to claim 18, wherein the moisture wicking layer is according to claim 1.

20. A garment having a gusset according to claim 16.
